(19) United States
(12) Patent Application Publication
(10) Pub. No.: US 2025/0249146 A1
(43) Pub. Date: Aug. 7, 2025

(54) FUNCTIONALIZED FIBER NETWORKS UTILIZING mRNA-RELEASING TOPOGRAPHICAL GUIDANCE CUES FOR NERVE REGENERATION

(71) Applicant: Rensselaer Polytechnic Institute, Troy, NY (US)

(72) Inventors: Ryan GILBERT, Wynantskill, NY (US); Devan PUHL, Troy, NY (US); Jessica FUNNELL, Green Island, NY (US); Tanner FINK, Troy, NY (US); Runye Helen ZHA, Troy, NY (US)

(73) Assignee: Rensselaer Polytechnic Institute, Troy, NY (US)

(21) Appl. No.: 18/856,193

(22) PCT Filed: Apr. 11, 2023
(86) PCT No.: PCT/US2023/018154

§ 371 Date: Oct. 11, 2024

Related U.S. Application Data
(60) Provisional application No. 63/329,770, filed on Apr. 11, 2022.
(60) Provisional application No. 63/458,513, filed on Apr. 11, 2023.

(51) Int. Cl. A61L 27/26; A61L 27/34; A61L 27/54; C08L 5/02; C08L 67/04; C08L 79/02; C12N 15/88

(52) U.S. Cl. CPC A61L 27/26; A61L 27/34; A61L 27/54; C08L 5/02; C08L 67/04; C08L 79/02; C12N 15/88; A61L 2300/258; A61L 2430/32

(57) ABSTRACT

Therapeutic agents such as mRNA fragments are delivered to a patient to treat peripheral nerve system injuries via functionalized fiber networks. The fiber networks are electrospun from poly(L-lactic acid), producing a hollow conduit of aligned fibers. The networks are functionalized with an anionic layer, e.g., dextran sulfate sodium salt (DSS), poly(3,4-dihydroxy-L-phenylalanine) (pDOPA). mRNA complexes including a cationic liposomal transfection portion and an mRNA portion, such as Ψ-modified mRNA encoding neurotrophin-3 (NT-3), are immobilized thereon. The fiber networks are positioned to bridge a peripheral nerve gap in the patient. These networks upregulate secretion of expressed oligopeptides, e.g., NT-3, by surrounding cells, and thus enhance neurite outgrowth. The networks serve as versatile non-viral mRNA delivery platforms. The mRNA transcript, gene delivery vehicle, and surface coatings can be modified to regulate different desired proteins, optimize transfection efficiency, enable targeted delivery, and tune loading efficiency and release kinetics.

### CROSS REFERENCE TO RELATED APPLICATION(S)

This application is a national stage filing of International Patent Application No. PCT/US2023/018154, filed Apr. 11, 2023, which claims the benefit of U.S. Provisional Application Nos. 63/329,770, filed Apr. 11, 2022, and 63/458,513, filed Apr. 11, 2023, which are incorporated by reference as if disclosed herein in their entirety.

### STATEMENT REGARDING FEDERALLY SPONSORED RESEARCH AND DEVELOPMENT

This invention was made with government support under grant number NS092754, awarded by the National Institutes of Health, grant number RX003502-01A1, awarded by the United States Department of Veterans' Affairs, and grant numbers 2045510 and DGE-1744655, awarded by the National Science Foundation. The government has certain rights in the invention.

### BACKGROUND

Peripheral nervous system (PNS) injury resulting from disease or trauma impacts over 20 million individuals in the United States alone and can significantly reduce the patient's quality of life. Injury to peripheral nerves either through diabetic complications, through tumor excisions, or through battlefield trauma destroys peripheral axons. Recovery from PNS injury is often incomplete due to limited regeneration of damaged peripheral axons and deficient reinnervation of surrounding tissues.

A complete nerve transection injury gap larger than 1 cm is typically addressed via surgical placement of a bridging graft to support axon regeneration and functional recovery. A nerve autograft or allograft are the current gold standard surgical options to bridge a large injury gap in a peripheral nerve. However, sural nerve autografts are difficult to obtain due to diabetic complications, and sural nerve harvest leads to donor site morbidity. There are several FDA-approved biomaterial constructs including tubes made from collagen or polymer that confine and then direct subsequent axonal regeneration, as well as one FDA-approved decellularized allograft product that can also be used to bridge peripheral nerve gaps up to 5 cm in length. However, these biomaterial constructs lack sufficient extracellular matrix (ECM) with the growth factors needed to promote robust regeneration. Further, allografts require extensive decellularization processes, and it is challenging to develop size-appropriate allografts for patients.

Delivery of neurotrophic factor proteins from biomaterials following nervous system injury is of interest due to their known benefits on the regeneration process. Neurotrophin-3 (NT-3) is a neurotrophic protein that binds to tyrosine kinase receptors present on cells throughout the PNS, including Schwann cells and dorsal root ganglia (DRG) neurons. This enables NT-3 to elicit a broad range of responses in vitro and in vivo, such as increasing Schwann cell migration, mediating Schwann cell myelination, enhancing neurite outgrowth from DRG explants, and improving sensory axon regeneration. Direct treatment with NT-3 protein supports PNS and central nervous system regeneration, but clinical translation is limited by the protein's short half-life (1.28±0.07 min) and thus its repeated administration for effective treatment. As an alternative, Ad and AAV viral vectors have been used to upregulate the production of NT-3 over a sustained period to increase neurite outgrowth in vitro and improve peripheral nerve regeneration in vivo. However, Ad and AAV therapeutics are limited by packaging capacity and long-term safety concerns related to immunogenicity or insertional mutagenesis.

Investigations into mRNA therapeutics are growing rapidly and can potentially overcome limitations of protein and DNA therapeutics, such as short half-life and insertional mutagenesis. mRNA therapeutics provide a non-viral approach to enable transient expression with high efficiency in hard-to-transfect primary PNS cells. Still, the instability and immunogenicity of mRNA and the challenge of delivering an efficacious dose to a target location has slowed development of mRNA therapeutics for tissue engineering and regenerative medicine. Electrospun fibers have been used to locally deliver therapeutic molecules while providing structural guidance cues, but a fibrous platform has yet to be developed to deliver therapeutic mRNA, e.g., for neural repair applications.

### SUMMARY

Aspects of the present disclosure are directed to a method of making a functionalized fiber network. In some embodiments, the method includes electrospinning a fiber network, the fiber network including one or more aligned fibers, preparing mRNA complexes including a cationic transfection portion and an mRNA portion, immobilizing mRNA complexes on the fiber network to form a functionalized fiber network. In some embodiments, immobilizing mRNA complexes on the fiber network to form a functionalized fiber network includes contacting the fiber network with a solution. In some embodiments, the solution includes 0.5 mg/mL DSS, 2 mg/mL pDOPA, or combinations thereof. In some embodiments, the method comprises plasma treating the fiber network prior to immobilizing the mRNA complexes thereon.

In some embodiments, the functionalized fiber network includes an anionic layer disposed between the aligned fibers and the mRNA complexes. In some embodiments, the fibers include poly(L-lactic acid), poly(L-lactic acid) derivatives, or combinations thereof. In some embodiments, the anionic layer includes dextran sulfate sodium salt (DSS), poly(3,4-dihydroxy-L-phenylalanine) (pDOPA), or combinations thereof. In some embodiments, the cationic transfection portion includes a liposomal transfection agent. In some embodiments, the mRNA portion encodes neutrophin-3 (NT-3). In some embodiments, the mRNA portion includes Ψ-modified mRNA encoding NT-3. In some embodiments, the fiber network is a hollow conduit having a lumen, wherein the mRNA complexes are immobilized within the lumen. In some embodiments, the functionalized network further includes one or more additional surface coatings, wherein mRNA complexes are incorporated in the additional surface coating.

Aspects of the present disclosure are directed to a functionalized fiber network. In some embodiments, an electrospun fiber network, the fiber network including one or more aligned fibers. In some embodiments, a plurality of mRNA complexes are immobilized on the electrospun fiber network, the mRNA complexes including a cationic transfection portion and an mRNA portion. In some embodiments, an anionic layer is disposed between the aligned fibers and the mRNA complexes.

In some embodiments, the fiber network includes poly(L-lactic acid), poly(L-lactic acid) derivatives, or combinations thereof. In some embodiments, the cationic transfection portion includes a liposomal transfection agent. In some embodiments, the mRNA portion encodes NT-3. In some embodiments, the mRNA portion includes Ψ-modified mRNA encoding NT-3. In some embodiments, the anionic layer includes dextran sulfate sodium salt (DSS), poly(3,4-dihydroxy-L-phenylalanine) (pDOPA), or combinations thereof. In some embodiments, the fiber network is a hollow conduit having a lumen, wherein the mRNA complexes are immobilized within the lumen. In some embodiments, the functionalized network further includes at least one additional surface coating, wherein mRNA complexes are incorporated in the additional surface coating.

Aspects of the present disclosure are directed to a method of delivering a therapeutic agent to a patient. In some embodiments, the method includes electrospinning a fiber network, the fiber network including one or more aligned fibers, preparing mRNA complexes including a cationic liposomal transfection portion and an mRNA portion including Ψ-modified mRNA encoding NT-3, immobilizing mRNA complexes on the fiber network to form a functionalized fiber network, providing the functionalized fiber network as a hollow conduit to a peripheral nerve gap in the patient, and taking up the mRNA from the functionalized network by one or more cells at or proximate the site of the peripheral nerve gap. In some embodiments, the functionalized fiber network includes an anionic layer disposed between the aligned fibers and the mRNA complexes. In some embodiments, the fibers include poly(L-lactic acid), poly(L-lactic acid) derivatives, or combinations thereof and the anionic layer includes DSS, pDOPA, or combinations thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a chart of a method of making a functionalized fiber network according to some embodiments of the present disclosure;

FIG. 2A is a schematic representation of a functionalized fiber network according to some embodiments of the present disclosure;

FIG. 3 is a chart of a method of delivering a therapeutic agent to a patient according to some embodiments of the present disclosure;

FIG. 4 is a schematic representation of whole dorsal root ganglia (DRG) explant and Schwann cell co-culture with functionalized fiber networks according to some embodiments of the present disclosure;

FIG. 5A is a graph showing Fourier transform infrared (FTIR) spectra for poly(L-lactic acid) (PLLA) fiber networks and fiber networks consistent with embodiments of the present disclosure;

FIG. 5B is a graph showing static water contact angles for PLLA fiber networks and functionalized fiber networks according to some embodiments of the present disclosure;

FIG. 5C is a graph showing zeta potentials for PLLA fiber networks and fiber networks consistent with embodiments of the present disclosure;

FIG. 6A shows scanning electron microscopy (SEM) images of PLLA fiber networks and fiber networks consistent with embodiments of the present disclosure;

FIG. 6B is a graph showing fiber diameter of PLLA fiber networks and fiber networks consistent with embodiments of the present disclosure;

FIG. 6C is a graph showing fast Fourier transform (FFT) analysis of PLLA fiber networks;

FIG. 6D is a graph showing fiber coverage electrospun PLLA fiber networks;

FIG. 7A is a graph showing loading efficiency of mRNA complexes for functionalized fiber networks consistent with embodiments of the present disclosure;

FIG. 7B is a graph showing release profiles of mRNA complexes for functionalized fiber networks consistent with embodiments of the present disclosure;

FIG. 8A are confocal microscopy images confirming adhesion of Schwann cells in the presence of functionalized fiber networks consistent with embodiments of the present disclosure;

FIG. 8B is a graph showing the concentration of Schwann cells on functionalized fiber networks consistent with embodiments of the present disclosure;

FIG. 9 is a series of graphs showing secretion of neurotrophin-3 (NT-3) protein by Schwann cells on functionalized fiber networks consistent with embodiments of the present disclosure;

FIG. 10A is a confocal microscopy image of primary rate whole DRG explants following culture in the presence of primary rate Schwann cells on PLLA fiber networks and fiber networks consistent with embodiments of the present disclosure;

FIG. 10B is a graph showing neurite extension following culture in the presence of primary rate Schwann cells on PLLA fiber networks and fiber networks consistent with embodiments of the present disclosure; and

FIG. 10C is a graph showing neurite area following culture in the presence of primary rate Schwann cells on PLLA fiber networks and fiber networks consistent with embodiments of the present disclosure.

### DESCRIPTION

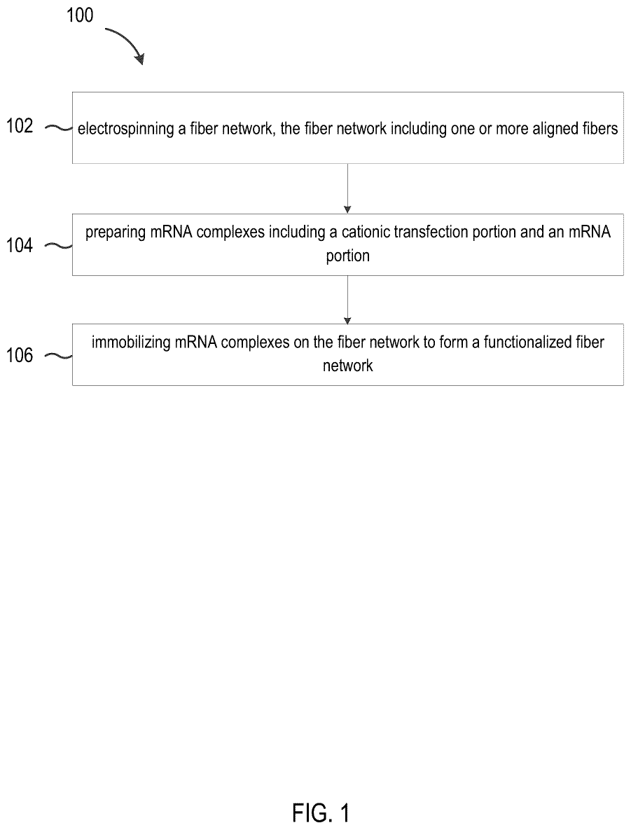

Referring now to FIG. 1, some embodiments of the present disclosure are directed to a method 100 of making a functionalized fiber network. At 102, a fiber network is electrospun. In some embodiments, the fiber network is electrospun 102 via metered flow through a spinneret to a rotating grounded collector, with a voltage applied therebetween. Design details related to apparatus suitable for performing electrospinning 102, e.g., spinneret design, collector design, etc., are within the capabilities of those of skill in the art. In some embodiments, the fiber network produced by electrospinning step 102 includes one or more aligned fibers. In some embodiments, the one or more aligned fibers are arranged longitudinally. In some embodiments, the fibers in the fiber network are composed of one or more polymers. In some embodiments, the one or more polymers are suitable for use in in vitro processes including one or more cells lines. In some embodiments, the one or more polymers are biocompatible. In some embodiments, the one or more polymers are suitable for use in vivo, e.g., implanting in a human patient. In some embodiments, the fibers include poly(L-lactic acid) (PLLA), poly(L-lactic acid) derivatives, or combinations thereof. In some embodiments, the fiber network is a substantially flat sheet. In some embodiments, the fiber network is a hollow conduit having a lumen. In some embodiments, the fiber network includes coaxially electrospun fibers. In some embodiments, the coaxially electrospun fibers have one or more outer layers having a first composition that surround a core having a second composition.

At 104, one or more mRNA complexes are prepared. In some embodiments, the mRNA complexes include a cationic transfection portion and an mRNA portion. In some embodiments, the cationic transfection portion is configured to bind the mRNA portion to the fiber network and facilitate uptake of the mRNA portion into one or more cells at or proximate to the network, as will be discussed in greater detail below. In some embodiments, the cationic transfection portion includes a liposomal transfection agent, e.g., JetIESSENGER® (Polyplus-transfection SA, Illkirch, FRANCE). In some embodiments, the mRNA portion includes one or more mRNA fragments that encode desired oligopeptides. In some embodiments, the mRNA includes one or more features for enhancing gene delivery and incorporation into cells, such as capping structures, modified nucleotides like pseudouridine-5′-triphosphate (Ψ), etc., or combinations thereof. In some embodiments, the mRNA portion encodes neutrophin-3 (NT-3). In some embodiments, the mRNA portion includes Ψ-modified mRNA encoding NT-3.

At 106, one or more mRNA complexes are immobilized on the fiber network to form a functionalized fiber network. In some embodiments, mRNA complexes are immobilized 106 via adsorption. In some embodiments, mRNA complexes are immobilized 106 directly on the fiber network. In some embodiments, the mRNA complexes are immobilized 106 on the fiber network via one or more linkers. Where the fiber network is a hollow conduit having a lumen, in some embodiments, the mRNA complexes are immobilized 106 within the lumen. Effective cationic transfection agents can condense the anionic genetic cargo into nanoparticles with a hydrodynamic size ranging from ˜50-200 nm while imparting a slight positive charge onto the particle.

In some embodiments, the fiber network includes one or more anionic layers disposed thereon. In some embodiments, mRNA complexes are immobilized 106 on the fiber network via an anionic layer, e.g., are adsorbed to an anionic layer. Thus, in some embodiments, the functionalized fiber network includes an anionic layer disposed between aligned fibers and mRNA complexes, as will be discussed in greater detail below. In some embodiments, the mRNA complexes are immobilized 106 on the fiber network via one or more linkers bound between the mRNA complexes and an anionic layer. Where the fiber network is a hollow conduit having a lumen, in some embodiments, an anionic layer is disposed on an inner surface, i.e., within the lumen. In some embodiments, the anionic layer includes dextran sulfate sodium salt (DSS), poly(3,4-dihydroxy-L-phenylalanine) (pDOPA), or combinations thereof. In some embodiments, the fiber network includes a plurality of anionic layers. In some embodiments, the anionic layers include a concentration of polyethylene glycol (PEG).

In some embodiments, immobilizing 106 mRNA complexes on the fiber network to form a functionalized fiber network includes contacting the fiber network with a solution including one or more anionic species. In some embodiments, the solution includes 0.5 mg/mL DSS; 2 mg/mL pDOPA, or combinations thereof. In some embodiments, the fiber network is plasma treated prior to immobilizing 106 the mRNA complexes thereon. In some embodiments, the functionalized network further includes one or more additional surface coatings, as will be discussed in greater detail below. In some embodiments, mRNA complexes are incorporated into the additional surface coating.

The fiber networks of the present disclosure effectively hold the mRNA complexes until they are positioned in a target environment, where the mRNA complexes are then released. The mRNA cargo is then taken up by the cells in that target environment, enhancing expression of that mRNA and thus production of the desired proteins, as will be discussed in greater detail below.

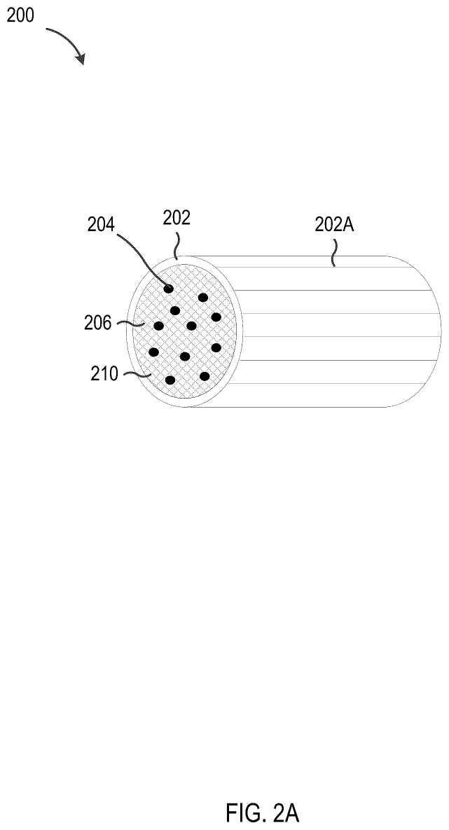

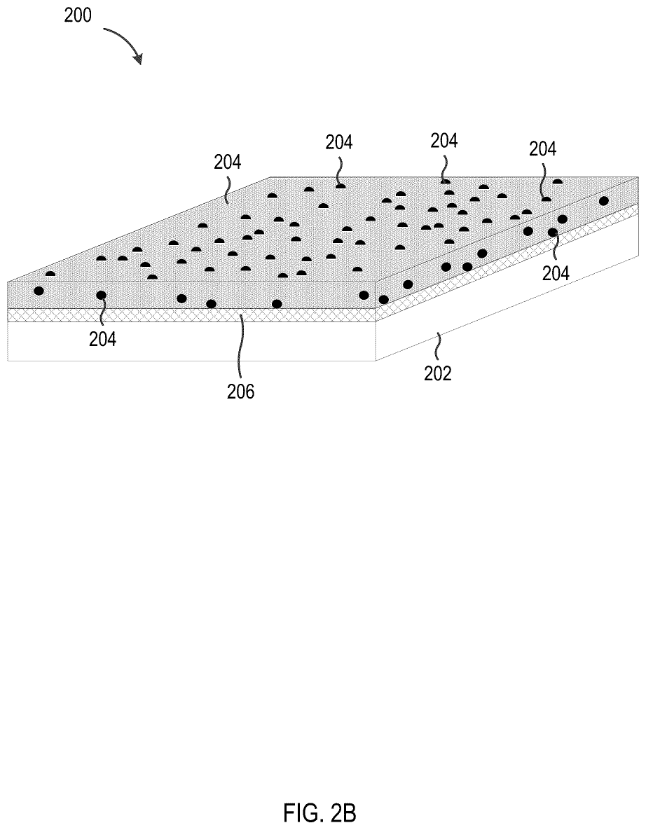

Referring now to FIG. 2A, some embodiments of the present disclosure are directed to a functionalized fiber network 200. In some embodiments, functionalized fiber network 200 includes one or more aligned fibers 202A forming a fiber network 202. In some embodiments, fiber network 202 includes a plurality of longitudinally oriented fibers 202A. As discussed above, in some embodiments, aligned fibers 202A are electrospun. In some embodiments, aligned fibers 202A are composed of one or more polymers. In some embodiments, aligned fibers 202A are composed of PLLA, PLLA derivatives, or combinations thereof.

In some embodiments, a plurality of mRNA complexes 204 are immobilized on fiber network 202. As discussed above, in some embodiments, mRNA complexes 204 include a cationic transfection portion and an mRNA portion. In some embodiments, the cationic transfection portion includes a liposomal transfection agent. In some embodiments, mRNA portion 204 encodes NT-3. In some embodiments, mRNA portion 204 includes Ψ-modified mRNA encoding NT-3.

In some embodiments, an anionic layer 206 disposed between fiber network 202, e.g., aligned fibers 202A, and mRNA complexes 204. In some embodiments, anionic layer 206 includes DSS, pDOPA, or combinations thereof. Immobilization of cationic gene delivery vehicles, e.g., mRNA complexes 204, to fiber network 202 fibers may occur through electrostatic interactions, hydrophobic interactions, hydrogen bonding, Schiff base or Michael addition reactions, or any other suitable interaction. By way of example, pDOPA possesses carboxyl groups and reactive o-quinones that support the immobilization of cationic delivery vehicles carrying genetic material through a variety of possible physical and chemical interactions, including electrostatic interactions, hydrophobic interactions, hydrogen bonding, or Schiff base or Michael addition reactions, depending on the surface chemistry of the gene delivery vehicle. DSS possesses sulfonate groups enabling immobilization of a cationic enzyme via electrostatic interactions.

Referring now to FIG. 2B, in some embodiments, functionalized network 200 further includes at least one additional surface coating 208. In some embodiments, mRNA complexes 204 are incorporated in additional surface coating 208.

Referring again to FIG. 2A, in some embodiments, functionalized fiber network 200 is a hollow conduit having a lumen 210. In some embodiments, mRNA complexes 204 are immobilized within lumen 210, i.e., on an interior wall of network 200. Functionalized fiber networks 200 having the hollow conduit conformation are particularly advantageous for use in peripheral nerve injuries. The networks 200 can be sized to fit around distal and proximal stumps of the peripheral nerve. While the fiber network directs migration of machinery for axonal regeneration between the two ends of the peripheral nerve gap, that machinery encounters concentrations of mRNA complexes that are taken up and enhance expression of beneficial proteins at the gap, e.g., NT-3, thereby helping to facilitate the regeneration. However, the present disclosure is not intended to be limiting in this regard, as one of ordinary skill in the art would understand that functionalized fiber network 200 may take on alterative conformations, e.g., flat sheets (see, e.g., FIG. 2B), curved sheets, angled sheets, or combinations thereof, and further that mRNA complexes 204 may be immobilized on any suitable surface of the network in order to best deliver mRNA at a desired concentration to an environment or location.

In some embodiments, functionalized fiber network 200 includes coaxially electrospun fibers (not pictured). In some embodiments, the coaxial electrospun fibers include mRNA complexes 204 incorporated into the core thereof. In some embodiments, functionalized fiber network 200 includes one or more additional components such as small molecule drugs, proteins, nucleic acids, additional therapeutically active compound, diluent, carrier molecule, adjuvant, excipient, etc., or combinations thereof.

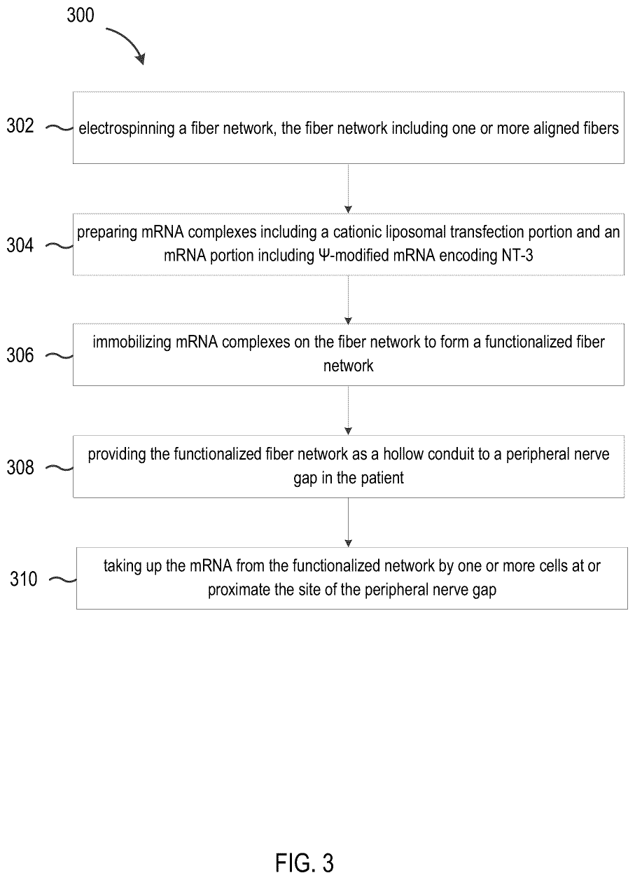

Referring now to FIG. 3, some embodiments of the present disclosure are directed to a method 300 of delivering a therapeutic agent to a patient. In some embodiments, at 302, a fiber network is electrospun. As discussed above, in some embodiments, the fiber network includes one or more aligned fibers. In some embodiments, the fibers include one or more polymers. In some embodiments, the fibers include PLLA, PLLA derivatives, or combinations thereof. In some embodiments, the therapeutic agent includes one or mRNA fragments, small molecule drugs, proteins, nucleic acids, additional therapeutically active compound, diluent, carrier molecule, adjuvant, excipient, etc., or combinations thereof.

In some embodiments, at 304, a plurality of mRNA complexes are prepared. As discussed above, in some embodiments, the mRNA complexes include a cationic liposomal transfection portion and an mRNA portion. In some embodiments, the mRNA portion includes Ψ-modified mRNA encoding NT-3. In some embodiments, the functionalized fiber network includes an anionic layer disposed between the aligned fibers and the mRNA complexes. In some embodiments, the anionic layer includes DSS, pDOPA, or combinations thereof. At 306, the mRNA complexes are immobilized on the fiber network to form a functionalized fiber network.

At 308, the functionalized fiber network is provided to a target site in a patient. In some embodiments, the functionalized fiber network is provided 308 to the site of an injury in the patient. In some embodiments, the functionalized fiber network is provided 308 to a peripheral nerve gap in the patient. In some embodiments, the functionalized fiber network is provided 308 to a peripheral nerve gap as a hollow conduit. At 310, the mRNA from the functionalized network are taken up by one or more cells at or proximate the site of the injury. In some embodiments, the mRNA dissociates into the environment where it is taken up by the cells. In some embodiments, the mRNA complex dissociates into the environment where it is taken up by the cells. In some embodiments, the mRNA are taken up by the cells directly from a surface of the functionalized fiber network. In some embodiments, the mRNA complex are taken up by the cells directly from a surface of the functionalized fiber network, where the mRNA subsequently dissociates from the complex in the cytoplasm, e.g., cytosol. NT-3 is a growth factor capable of enhancing axon regeneration and inducing Schwann cell plasticity. However, NT-3 is down-regulated in the distal nerve after sciatic nerve transection. The fiber networks of the present disclosure exhibit enhanced delivery of mRNA to endogenous cells within the environment surrounding the network, and further result in enhanced expression of that mRNA at or proximate the site of an injury. By way of example, enhanced expression of NT-3 by cells at a peripheral nerve gap significantly promotes peripheral axon regeneration for functional recovery.

### EXAMPLE

NT-3 mRNA was synthesized. Briefly, a pcDNA3.1(+) expression vector encoding rat NT-3 was expanded in bacteria and then isolated and purified via a maxi-prep. Next, the pcDNA3.1(+)_NT-3 was linearized, and the template DNA was amplified using standard polymerase chain reaction (PCR). Finally, Ψ-modified, anti-reverse cap analog (ARCA)-capped mRNA encoding NT-3 was synthesized via in vitro transcription and purified using spin columns. The NT-3 mRNA quality and bioactivity were evaluated via a bioanalyzer and enzyme-linked immunosorbent assay (ELISA), respectively.

mRNA/JetMESSENGER® lipoplexes were formed. Briefly, anionic NT-3mRNA and cationic JetMESSENGER® were complexed at a mass-to-volume ratio of 1 μg NT-3mRNA to 2 μL JetMESSENGER® (1:2 w/v) in the provided mRNA buffer. The solution was mixed thoroughly and incubated for 15 min at room temperature before use. The hydrodynamic size, polydispersity index, and charge of the formed mRNA lipoplexes were evaluated by an Anton Paar Litesizer™ 500 using dynamic light scattering (DLS) and measuring the zeta potential of particles in suspension. Three solutions of each mRNA type were prepared and analyzed (n=3).

Commercially produced CleanCap® Enhanced Green Fluorescent Protein mRNA (eGFPmRNA), which is similar in size to the NT-3mRNA (˜1 kb), was complexed to JetMESSENGER® and characterized in the same manner described above. eGFPm-RNA/JetMESSENGER® lipoplexes were used to conduct bolus transfection efficiency experiments and the eGFPmRNA immobilization pilot study for preliminary investigation of mRNA-loaded electrospun fiber design.

PLLA films and aligned PLLA fibers were fabricated onto glass coverslips using drop-casting and electrospinning techniques. Briefly, a 4% (w/w) solution of PLLA in chloroform was prepared, and 50 μL or 300 μL of the solution was drop cast evenly onto 15 mm×15 mm or 24 mm×50 mm glass coverslips, respectively. The 15 mm×15 mm films were used for contact angle characterization or before electrospinning fibers to secure the fibers to the coverslip and ensure that cells cultured onto the fibers only encountered one material type. The 24 mm×50 mm films were used for zeta potential characterization. Next, a 12% (w/w) solution of PLLA in chloroform was prepared and poured into a 5 mL syringe with a 22 G×1.5 inch needle and loaded into a syringe pump. The 15 mm×15 mm PLLA film-coated coverslips were attached to a grounded wheel (1 cm thickness, 22 cm diameter). Highly aligned electrospun fibers were fabricated onto the PLLA films using a vertical electrospinner and the following electrospinning parameters: 15 min collection time, 15 kV applied voltage, 1500 rpm wheel rotation speed, 4 cm collection distance, 2 mL/h flow rate, and 23±1% relative humidity. At least three 4% and 12% PLLA solutions were prepared to fabricate films and aligned PLLA fibers in material triplicate for each experiment (n=3).

All films that would undergo surface characterization (contact angle and zeta potential analysis) and all electrospun fiber scaffolds were dip-coated in a 4% solution of PLLA in chloroform on all four edges to ensure that the polymer scaffold did not lift from the glass coverslip during the biomaterial surface coating process or experiments that followed.

Surface coatings were investigated to improve mRNA immobilization and subsequent mRNA delivery to cells. PLLA films and electrospun fibers were sterilized under UV for 30 min. Immediately before coating, the films and fibers were plasma-treated with environmental air on the medium setting for 1 min with venting every 10 sec using an Expanded Plasma Cleaner to improve surface hydrophilicity. Briefly, the coating solutions included 1) DSS and sodium chloride (NaCl) in sterile 1×PBS or 2) 3,4-dihydroxy-L-phenylalanine (L-DOPA), N,N-Bis(2-hydroxyethyl)glycine (BICINE), and NaCl in sterile deionized water (diH₂O) adjusted to pH 8.5 with sodium hydroxide (NaOH). The 15 mm×15 mm PLLA films or fibers were placed flat in a sterile 12-well plate and submerged in 1.5 mL of the respective coating solution, while the 24 mm×50 mm PLLA films were placed flat in a 60-mm dish and submerged in 5.5 mL of the respective coating solution. The plate was then placed on a VWR mini orbital shaker (15 mm orbit) at 200 rpm to maintain gentle agitation of the coating solution throughout the coating duration. After coating, each scaffold was washed 3× with sterile diH₂O and dried overnight at 40° C. in a vacuum oven. Table 1 summarizes the DSS and pDOPA coating conditions to functionalize the PLLA surfaces selected for further material characterization and in vitro testing.

The 15 mm×15 mm uncoated PLLA and DSS- and pDOPA-coated electrospun fiber groups were removed from the glass coverslip and analyzed on a Nicolet™ iS5 FTIR Spectrometer to investigate the chemical properties of the fiber surface. The mean absorbance spectrum of each group was obtained from ˜500-4000 cm¹ and normalized to their respective base (minimum) and peak (maximum) values through minimum-maximum normalization. Three separate scaffolds were analyzed per group (n=3).

The static water contact angle on uncoated PLLA and DSS- and pDOPA-coated film groups was measured using a Kruss DSA100 Drop Shape Analyzer to investigate the surface wettability. Contact angle measurements were conducted on 15 mm×15 mm film samples rather than electrospun fibers, as the water droplet would spread directionally along the fibers introducing a confounding variable. A 3 μL droplet volume was used, and the angles at the liquid-vapor and solid-liquid interface were fit on the Drop Shape Analysis 4 software. Three droplets were analyzed on each film, averaging each measurement's left and right angles, and three separate films were analyzed per group (n=3).

The zeta potential of uncoated PLLA and DSS- and pDOPA-coated film samples was measured by an Anton Paar SurPASS™ 3 Electrokinetic Analyzer for solid surface analysis using an adjustable gap cell to determine the material surface charge. The 24 mm×50 mm uncoated PLLA and DSS- and pDOPA-coated film groups were removed from the glass and cut to 20 mm×10 mm to fit into the sample holder. All measurements were performed in a 10 mM KCl solution titrated to a physiologically relevant pH of 7.4 (7.469±0.178) using the instrument's automated titration system. Each run included five zeta potential measurements: however, only the last three measurements for each run were analyzed to ensure the system was under equilibrium. Three separate runs were performed for each film group (n=3).

The morphological features of the uncoated PLLA and 0.5DSS4h- and 2pDOPA4h-coated electrospun fiber groups were visually assessed and quantified to ensure consistency among batches and groups, as changes in these features can affect cell response. Uncoated PLLA and 0.5DSS4h- and 2pDOPA4h-coated PLLA fibers were sputter-coated with approximately 1 nm of Au/Pd using a Hummer V Technics sputter coater and then imaged via scanning electron microscopy (SEM) to visualize each fiber group. Images were captured on a Versa 3D™ Dual Beam™ SEM using an accelerating voltage of 2 kV. Images for morphological characterization were captured at a 2500× magnification, and representative images were captured at a 1000× magnification.

TABLE 1
Summary table of surface coatings.

| Coating Name | Coating Solution | Coating Time |
|---|---|---|
| 0.5DSS4 h | 0.5 mg/mL DSS, 1M NaCl, | 4 h |
| | 1x PBS | |
| 2pDOPA4 h | 2 mg/mL L-DOPA, 10 mM | 4 h |
| | BICINE, 250 mM NaCl, | |
| | diH₂O, pH 8.5 | |

Fiber alignment, fiber diameter, and percent fiber coverage were characterized via FIJI Software. The fiber alignment, diameter, and percent coverage of the uncoated PLLA fiber scaffolds were characterized, while the fiber diameters of the 0.5DSS4h- and 2pDOPA4h-coated were characterized. 100 fibers per replicate were analyzed to determine fiber diameter, and six fields of view per replicate were analyzed to determine fiber alignment and percent fiber coverage. At least one electrospun fiber scaffold per replicate and three separate replicates per fiber group were analyzed to characterize fiber alignment, fiber diameter, and percent fiber coverage (n=3).

A total of 3 μg of NT-3mRNA was immobilized per fiber scaffold to the uncoated PLLA and 0.5DSS4h- and 2pDOPA4h-coated fibers to load a dose large enough to remain efficacious over a sustained period. The uncoated PLLA and 0.5DSS4h- and 2pDOPA4h-coated fibers were sterilized via UV for 30 min. A 300 μL solution of Polyplus® mRNA buffer containing 3 μg of NT-3mRNA and 6 μL of JetMESSENGER® was pipetted onto each scaffold and incubated at 37° C. for 30 min. After the 30 min incubation, the immobilization lipoplex solution was removed, and each scaffold was washed with nuclease-free diH₂O. The mRNA immobilized groups will be referred to as PLLA mRNA, 0.5DSS4h mRNA, and 2pDOPA4h mRNA going forward. Immobilization solution containing only mRNA buffer (no added lipoplex) was also incubated with a separate set of uncoated PLLA fibers, and each scaffold was washed with nuclease-free diH₂O to serve as the negative control PLLA fiber group.

The Quant-iT™ RiboGreen™ RNA reagent kit was to quantify the amount of mRNA in solution and determine the mRNA loading efficiency and release kinetics. The standard curve was constructed by diluting a NT-3mRNA/JetMESSENGER® lipoplex solution to known mRNA concentrations. Highly negatively charged heparin sodium sulfate was added to the standard and unknown sample solutions at a final concentration of 20 μg/mL, mixed thoroughly, and incubated for 10 min at room temperature to decomplex the mRNA from the JetMESSENGER®. All decomplexed standard and sample solutions were mixed 1:1 with the Quant-iT™ RiboGreen™ RNA reagent, transferred into a black polystyrene 96-well assay plate, incubated for 5 min, and the concentration of mRNA was determined by measuring fluorescence on a Tecan infinite m200 microplate reader.

The amount of unbound NT-3mRNA was assessed using the Quant-iT™ RiboGreen™ RNA reagent kit to calculate the percent loading efficiency. Briefly, the control and experimental immobilization solutions and wash solutions were collected from the PLLA (negative control), PLLA mRNA, 0.5DSS4h mRNA, and 2pDOPA4h mRNA fibers. The collected samples were stored at −80° C. until quantified via Quant-iT™ RiboGreen™. The percentage of the 3 μg of NT-3mRNA initially loaded per scaffold present in the immobilization solution and wash solution was summed and subtracted from 100 to obtain the percent loading efficiency. Different immobilization lipoplex solutions were prepared to immobilize mRNA in each replicate, and six replicates were assessed per fiber group (n=6).

The effects of bolus transfection and the electrospun fiber-mediated mRNA delivery platforms on primary rat Schwann cells were investigated, as they are the principal glia of the PNS and vital to the peripheral nerve repair process. The animal procedures outlined were approved by the University of Miami's Institutional animal care and use committee (IACUC). The sciatic nerves from three-day-old Sprague Dawley rats (P3) were isolated, and Schwann cells were obtained and purified to >95% purity before cryopreservation. Before experimental culture, the Schwann cells were thawed and cultured for 7 days in 1:10 Poly(L-lysine)-coated T75 flasks in a cell culture incubator (5% CO₂ and 37° C.) with biweekly changes of Schwann cell media containing Dulbecco's Modified Eagle Medium (DMEM) supplemented with 10% fetal bovine serum, 1% penicillin-streptomycin, 1% GlutaMAX™, 3 μM forskolin, and 1.25 nM heregulin. Schwann cells expanded to passages three and four were used for all experiments.

Schwann cell adhesion on the uncoated PLLA and 0.5DSS4h- and 2pDOPA4h-coated fibers with and without immobilized NT-3mRNA/jetMESSENGER® lipoplexes was assessed to determine if the surface coatings or presence of lipoplexes affected cell adhesion. Primary rat Schwann cells were seeded at 100,000 cells per scaffold (˜444 cells/mm²) in Schwann cell media onto the following 15 mm×15 mm electrospun fiber scaffold groups: PLLA (negative control). 0.5DSS4h, 2pDOPA4h, PLLA mRNA, 0.5DSS4h mRNA, and 2pDOPA4h mRNA. The cells were cultured in a cell culture incubator (5% CO₂ and 37° C.) for 24 h. After 24 h, Schwann cell cultures were fixed with 4% (v/v) PEA in 1×PBS for 15 min, washed with 1×PBS three times, blocked with 0.1% (v/v) Triton™-x 100 and 5% (w/v) BSA in 1×PBS for 1 h, incubated with a 1:1000 dilution of DAPI nuclear stain in 1×PBS for 15 min, and then washed with 1×PBS three times. Three separate replicates were cultured for each fiber group (n=3).

Images were captured using MetaMorph® Premier 7.7.3.0 imaging software and a 289 Olympus IX-81 confocal microscope at a 20× magnification. FIJI software was used to process and analyze all images identically. The 2D maximum intensity projection of each image was obtained, and the background was subtracted using a rolling ball algorithm with a radius of 50 pixels. The number of cells per image was determined by semi-automatically thresholding each processed DAPI channel image and then using the analyze particles setting on FIJI software. The area of the field of view was determined for a 20× image. These values were used to determine the number of cells per area. Six fields of view were captured and analyzed per coverslip, and three separate coverslips were analyzed per fiber group (n=3).

Schwann cell NT-3 secretion into culture media was assessed using a rat NT-3 ELISA kit to determine if the electrospun fiber-mediated mRNA delivery platforms induced increased, sustained Schwann cell NT-3 secretion. Schwann cells were seeded at 100,000 cells per scaffold (˜444 cells/mm²) in Schwann cell media onto the following 15 mm×15 mm electrospun fiber scaffold groups: PLLA (negative control), PLLA mRNA, 0.5DSS4h mRNA, and 2pDOPA4h mRNA. In addition, Schwann cells cultured onto negative control PLLA fibers and transfected with bolus addition of lipoplex containing 1 μg NT-3mRNA to 2 μL JetMESSENGER® or 3 μg NT-3mRNA to 6 μL JetMESSENGER® per sample served as the positive control fiber groups and will be referred to as 1 μgBolus mRNA and 3 μgBolus mRNA going forward. The Schwann cells were cultured in a cell culture incubator (5% CO₂ and 37° C.), and culture media was collected from each sample and replaced at 1, 2, 3, 4, 5, 6, 7, 10, 14, and 21 days after seeding onto mRNA immobilized fibers or after the bolus transfection. The samples were centrifuged at 4° C. for 10 min at 1500 rcf to remove any debris and aliquoted and stored at −80° C. until analyzed. The amount of rat NT-3 protein was quantified using a rat NT-3 ELISA kit according to the manufacturer's protocol. Different immobilization lipoplex solutions were prepared to immobilize mRNA in each replicate, and four replicates were assessed per fiber group (n=4).

Dorsal root ganglia (DRG) explants were co-cultured in the same media environment as Schwann cells cultured directly onto the electrospun fiber-mediated mRNA delivery platforms to determine if these platforms induced increased Schwann cell secretion of NT-3 to levels significant enough to improve DRG neurite outgrowth. Co-culture media including a 1:1 ratio of DMEM to neurobasal media (DMEM:NBM) supplemented with 2% fetal bovine serum, 2% B-27®, 1% penicillin-streptomycin, and 250 mM GlutaMAX™ was prepared. Schwann cells were cultured at 100,000 cells per scaffold (˜444 cells/mm²) in the co-culture media onto the following 15 mm×15 mm aligned electrospun fiber scaffold groups: PLLA (negative control), PLLA mRNA, 0.5DSS4h mRNA, 2pDOPA4h mRNA, 1 μgBolus mRNA, and 3 μgBolus mRNA. Schwann cells were cultured in these conditions for 24 h in a cell culture incubator (5% CO₂ and 37° C.) before beginning co-culture experiments with whole DRG explants.

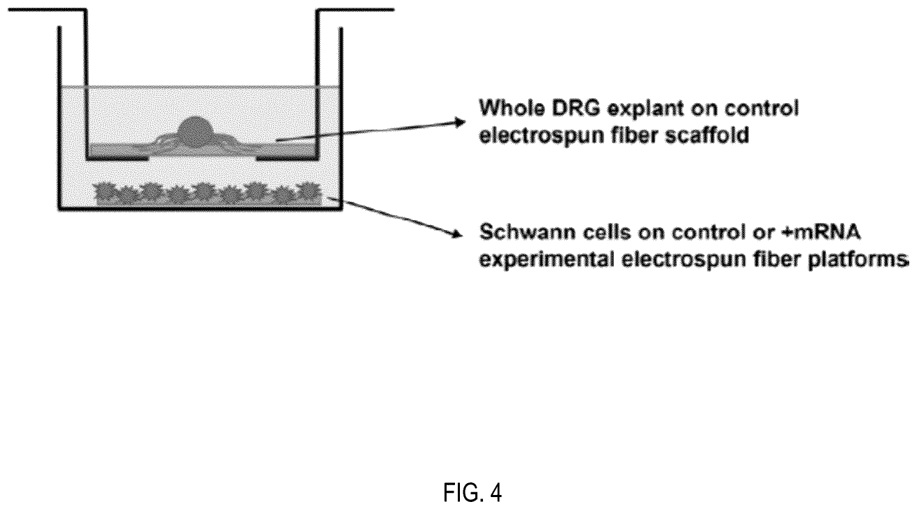

The animal procedures outlined were approved by the Rensselaer Polytechnic Institute's IACUC. Whole DRG explants were isolated from P2 Sprague Dawley rats according to previously described methods. Referring now to FIG. 4, a DRG explant and Schwann cell co-culture set-up was used in an attempt to mimic the signaling environment between Schwann cells and neurons following graft implantation and enables investigation of the effects of Schwann cell NT-3 secretion on DRG neurite outgrowth. Briefly, in-house 3D printed polylactic acid transwells were placed into each well containing the Schwann cell-seeded fibers. A separate set of negative control PLLA fibers were plasma-treated to improve DRG adhesion. Schwann cells were cultured on PLLA (negative control), PLLA mRNA, 0.5DSS4h mRNA, 2pDOPA4h mRNA, 1 μgBolus mRNA, and 3 μgBolus mRNA electrospun fiber platforms that were resting in the bottom of the well. Whole DRG explants were cultured on negative control PLLA electrospun fiber scaffolds submerged in the same media and suspended above the Schwann cell cultures via a polylactic acid transwell. The PLLA fibers were submerged in the conditioning co-culture media suspended above the Schwann cell-seeded fiber scaffolds by the transwells. Three whole DRG explants were cultured onto each PLLA fiber scaffold suspended in the transwell. The DRG explants were co-cultured with Schwann cells for 4 days in a cell culture incubator (5% CO₂ and 37° C.).

Confocal images of immunocytochemically-labeled DRG were captured and assessed to investigate neurite outgrowth. After the 4-day co-culture, the DRG explants were fixed, washed, and blocked. Next, the DRG were incubated overnight at 4° C. in a primary antibody solution containing 0.1% (v/v) TWEEN®-20 and 5% BSA with a 1:500 dilution of mouse polyclonal RT-97 primary antibody in 1×PBS. The following morning, the DRG were washed with 1×PBS three times and incubated in a secondary antibody solution containing 0.1% TWEEN®-20 and 5% BSA with a 1:1000 dilution of Alexa Fluor donkey anti-mouse 488 secondary antibody in 1×PBS for 1 h. Finally, the DRG were stained with DAPI nuclear stain and washed.

Images were captured at a 4× magnification and processed. Each processed whole DRG image was stitched using Photoshop CS2. Neurite outgrowth from DRG explants was assessed by quantifying neurite extension and area. The length of the ten longest neurites extending from the body of each DRG explant was measured using the line tool in FIJI software, and the average maximum neurite extension was calculated for each whole DRG explant. Next, the whole DRG body was excluded from each image, and the percent area covered by the neurites extending from the DRG explant body was quantified using a semi-automated thresholding FIJI plugin. Each whole DRG was considered an individual replicate, and 24 to 27 whole DRG were analyzed per culture condition (n=24-27).

Table 2 summarizes the control and experimental groups investigated. Table 3 summarizes the approximate size (hydrodynamic diameter), polydispersity, and surface charge (zeta potential) of the NT-3mRNA/JetMESSENGER® lipoplexes in suspension. Complexation of the NT-3mRNA or eGFPmRNA with JetMESSENGER® resulted in a monodisperse population of lipoplexes of similar size and charge, as evidenced by the overlapping size distribution and zeta potential distribution peaks. Bolus delivery of eGFPmRNA/JetMESSENGER® lipoplexes revealed JetMESSENGER® effectively delivered eGFPmRNA to Schwann cells with high efficiency, resulting in >95% of cells expressing eGFP over the first 3 days and 18.6±10.3% of cells still expressing detectable levels of eGFP by day 7 post-transfection.

TABLE 2
Summary table of control and experimental electrospun fiber groups

| | | Coating | | |
|---|---|---|---|---|
| Group Name | Biomaterial | Concentration | Coating Time | mRNA delivery |
| PLLA (Negative | Aligned PLLA | N/A | N/A | N/A |
| Control) | electrospun | | | |
| | fibers | | | |
| 0.5DSS4 h | Aligned PLLA | 0.5 mg/mL DSS | 4 h | N/A |
| | electrospun | | | |
| | fibers | | | |
| 2pDOPA4 h | Aligned PLLA | 2 mg/mL L- | 4 h | N/A |
| | electrospun | DOPA | | |
| | fibers | | | |
| PLLA mRNA | Aligned PLLA | N/A | N/A | mRNA |
| | electrospun | | | lipoplexes |
| | fibers | | | immobilized to |
| | | | | fiber surface |
| 0.5DSS4 h | Aligned PLLA | 0.5 mg/mL DSS | 4 h | mRNA |
| mRNA | electrospun | | | lipoplexes |
| | fibers | | | immobilized to |
| | | | | fiber surface |
| 2pDOPA4 h | Aligned PLLA | 2 mg/mL L- | 4 h | mRNA |
| mRNA | electrospun | DOPA | | lipoplexes |
| | fibers | | | immobilized to |
| | | | | fiber surface |
| 1 μgBolus | Aligned PLLA | N/A | N/A | Bolus addition |
| mRNA, | electrospun | | | of mRNA |
| | fibers | | | lipoplexes |
| | | | | carrying 1 μg |
| | | | | mRNA per well |
| 3 μgBolus | Aligned PLLA | N/A | N/A | Bolus addition |
| mRNA | electrospun | | | of mRNA |
| | fibers | | | lipoplexes |
| | | | | carrying 3 μg |
| | | | | mRNA per well |

TABLE 3
Characterization of approximate NT-3mRNA
lipoplex size, polydispersity, and charge

| | Hydrodynamic | Polydispersity | |
|---|---|---|---|
| Lipoplex | Diameter | Index | Zeta Potential |
| NT3mRNA/JetMESSENGER ® | 242.5 ± 32.6 nm | 15.0 ± 4.4% | 42.1 ± 1.3 mV |

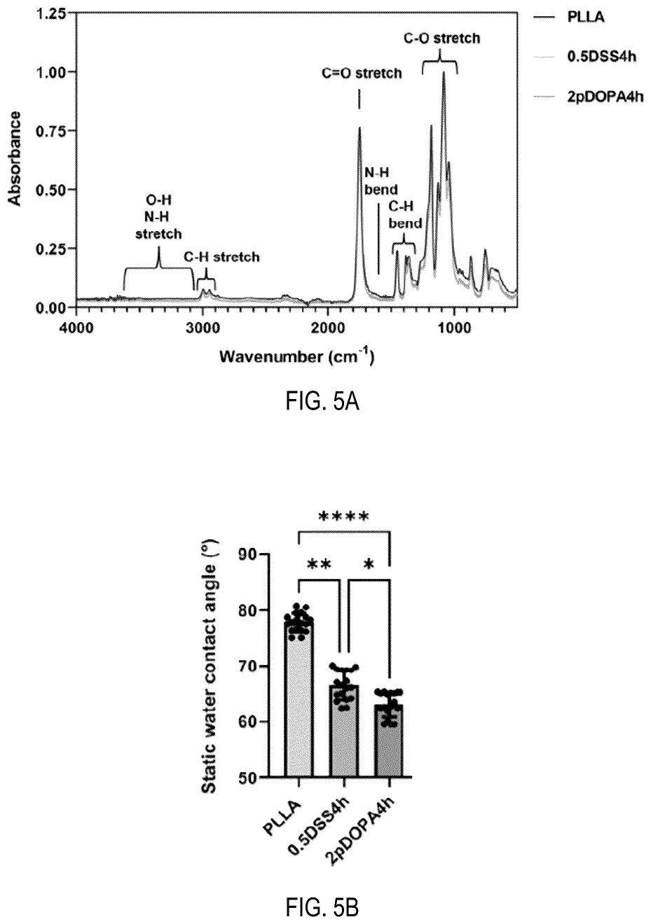

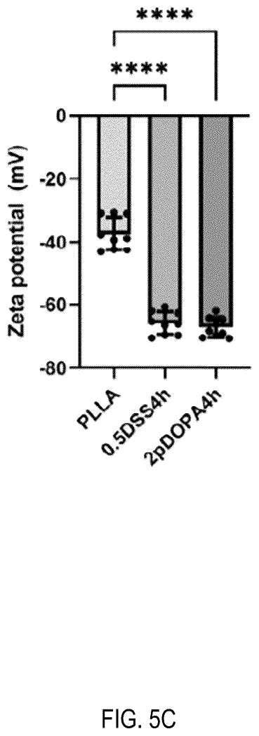

Referring now to FIGS. 5A-5C, the resulting Fourier transform infrared (FTTR) spectrum labeled with key identifying peaks and the contact angle and zeta potential data of the 0.5DSS4h- and 2pDOPA4h-coated experimental groups were compared to the uncoated PLLA control group. FTTR spectra revealed no noticeable change between the uncoated PLLA adsorption spectrum and the 0.5DSS4h- and 2pDOPA4h-coated fiber spectra (see FIG. 5A). The static water contact angles of the 0.5DSS4h (66.4±2.6°)- and 2pDOPA4h (63.0±2.2°)-coated film groups were significantly lower than the uncoated PLLA film group (77.8±1.70) (see FIG. 5B), indicating improved surface hydrophilicity of the coated groups. Additionally, the zeta potential of the 0.5DSS4h (−66.0±3.6 mV)- and 2pDOPA4h (−67.2±3.2 mV)-coated film groups was significantly more negative than the uncoated PLLA film group (37.4±5.5 mV) (see FIG. 5C), indicating the coated groups possessed a more negative surface charge.

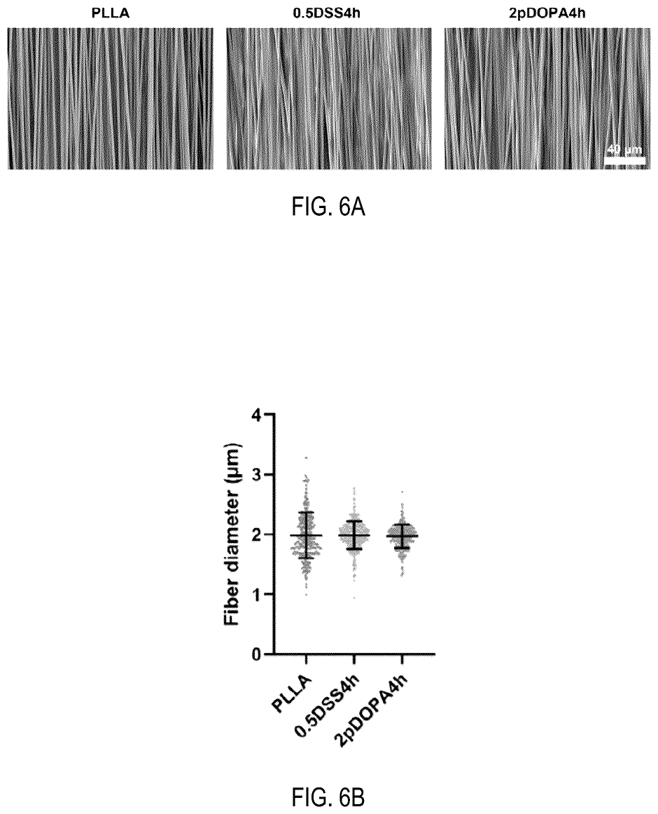

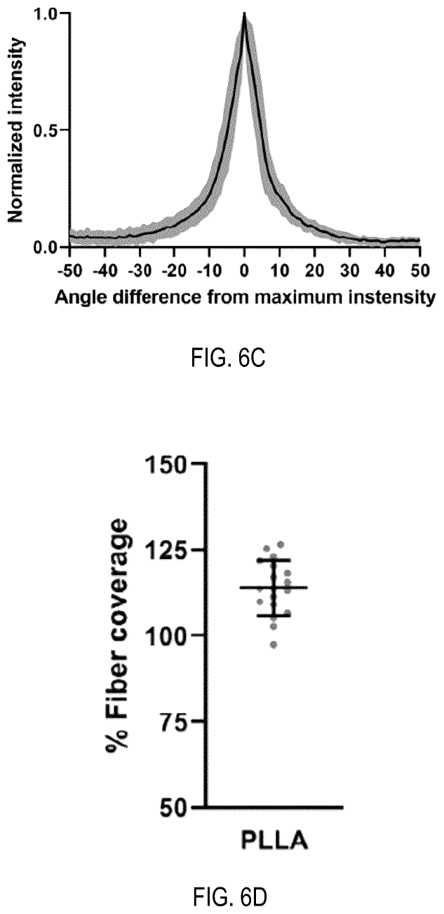

Referring now to FIGS. 6A-6D, SEM images of the uncoated PLLA and 0.5DSS4h- and 2pDOPA4h-coated fiber scaffolds showed no visible difference in fiber morphology (see FIG. 6A). Further, the mean fiber diameter of the uncoated PLLA fibers (1.98±0.38 pm) was statistically similar to the mean fiber diameter of the 0.5DSS4h (1.99±0.23 pm)- and 2pDOPA4h (1.96±0.20 pm)-coated fiber groups (p>0.05), indicating that the coatings did not alter fiber diameter (see FIG. 6B). The fast Fourier transform (FFT) analysis of the uncoated PLLA fiber group displayed high fiber alignment, illustrated by the sharp intensity peak with an area under the curve of 15.79±4.79 (see FIG. 6C). The PLLA fibers were collected at a near-monolayer density with some overlap, resulting in a percent fiber coverage over 100% (113.9±8.0%) (see FIG. 6D).

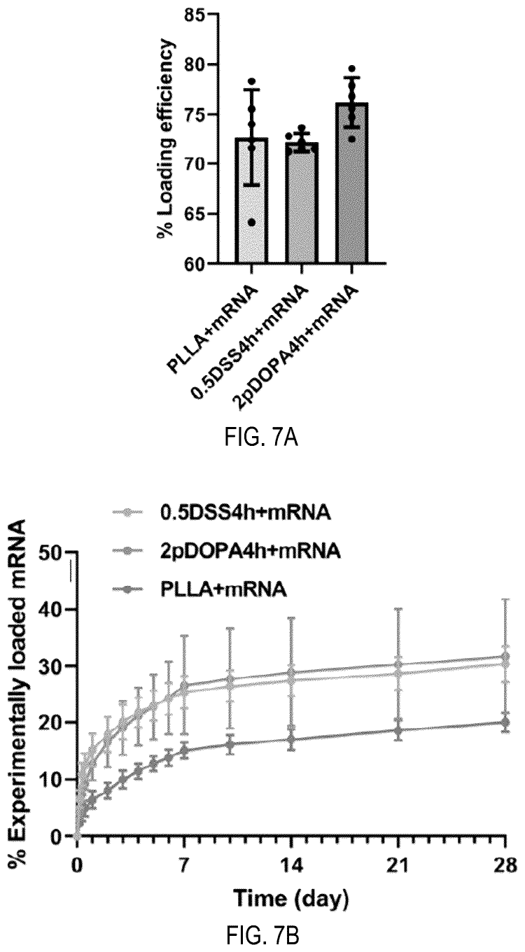

Referring now to FIGS. 7A-7B, the percent loading efficiencies (and resulting mass) of the initial 3 μg (3000 ng) of NT-3mRNA loaded per scaffold were similar amongst groups, resulting in 72.7±4.8% (2180±145 ng) for PLLA mRNA fibers, 72.2±1.0% (2165±29 ng) for 0.5DSS4h mRNA fibers, and 76.2±2.5% (2286±75 ng) for 2pDOPA4h mRNA fibers (see FIG. 7A). The 0.5DSS4h- and 2pDOPA4h-coated fibers groups exhibited an increased rate of NT-3mRNA release, particularly over the first 7 days, compared to the uncoated PLLA fiber group (see FIG. 7B). Based on the NT-3mRNA loading efficiency determined for the respective samples and replicates, the cumulative percent (and mass) of experimentally loaded NT-3mRNA released over 28 days from the PLLA mRNA, 0.5DSS4h mRNA, and 2pDOPA4h mRNA fiber groups were 20.0±1.7% (435±42 ng). 30.4±3.2% (658±65 ng), and 31.7±10.0% (726±236 ng), respectively (see FIG. 7B).

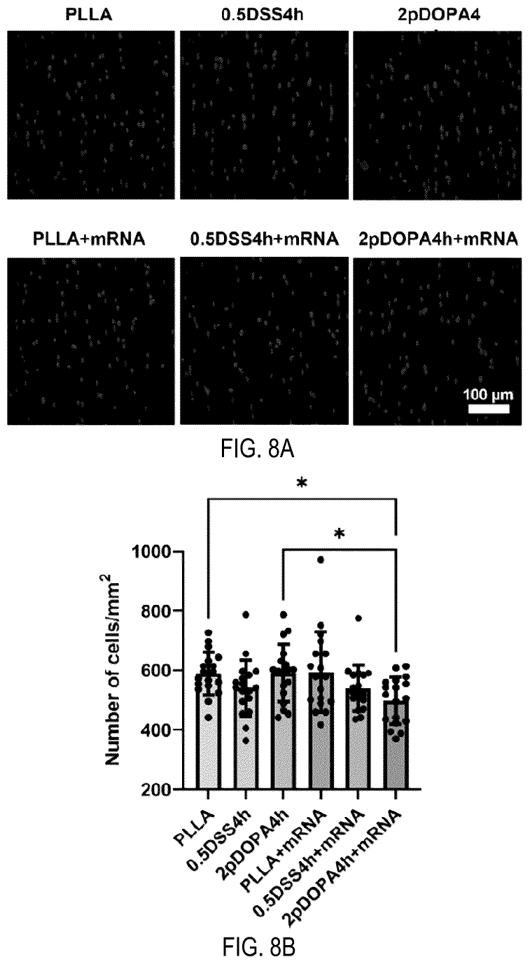

Referring now to FIG. 8A, confocal images were used to determine the number of adherent Schwann cells per mm² after 24 h. Schwann cell adhesion onto the uncoated PLLA (589±72 cells/mm²) fibers was similar to the 0.5DSS4h (541±94 cells/mm²)- and 2pDOPA4h (590±96 cells/mm²)-coated fiber groups (p>0.05), indicating the 0.5DSS4h and 2pDOPA4h coatings alone do not affect Schwann cell adhesion compared to the uncoated PLLA fibers. Similarly, Schwann cell adhesion to the PLLA mRNA (594±135 cells/mm²) and 0.5DSS4h mRNA (540±78 cells/mm²) fiber groups was similar to that observed in the non-mRNA immobilized fiber groups, indicating that the presence of mRNA on these fiber groups did not affect Schwann cell adhesion. Conversely, Schwann cell adhesion decreased in the 2pDOPA4h mRNA fiber group (498±79 cells/mm²) compared to the uncoated PLLA and 2pDOPA4h-coated fiber groups (see FIG. 8B), indicating the mRNA lipoplex presence on 2pDOPA4h mRNA fiber group influences Schwann cell adhesion.

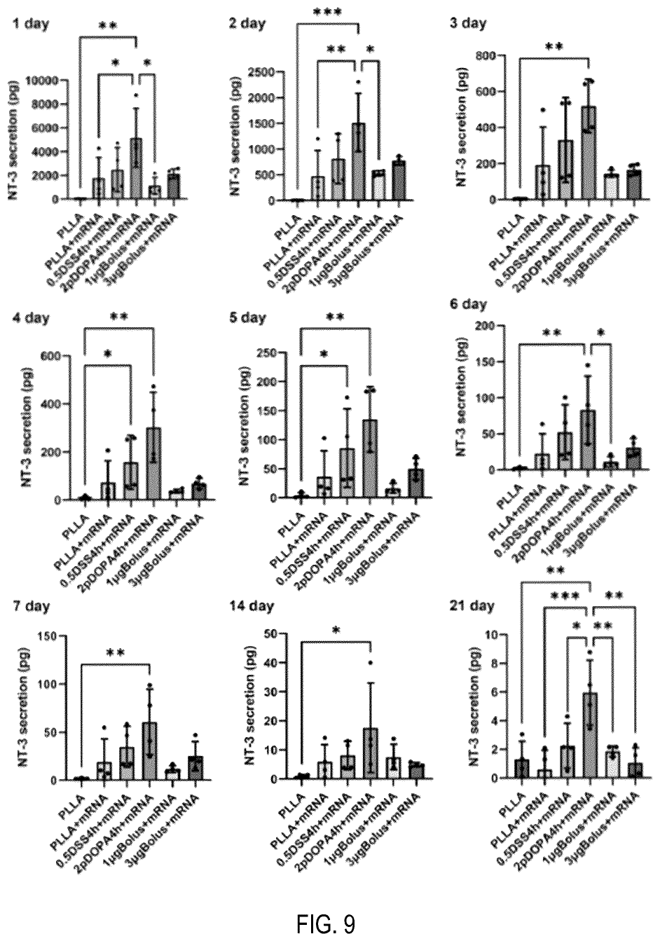

Referring now to FIG. 9, the differences in Schwann cell secretion of NT-3 protein into culture media was measured over 21 days. Schwann cells cultured on the 2pDOPA4h mRNA fibers secreted increased amounts of NT-3 protein 1, 2, 3, 4, 5, 6, 7, 14, and 21 days post-seeding compared to those cultured on the negative control PLLA fibers. Schwann cells cultured on the 2pDOPA4h mRNA fibers also secreted increased amounts of NT-3 protein 1, 2, and 21 days post-seeding compared to those cultured on the PLLA mRNA fibers; 1, 2, 6, and 21 days post-seeding compared to those cultured on the 1 μgBolus mRNA positive control fiber group; and 21 days post-seeding compared to those cultured on the 0.5DSS4h mRNA fibers or 3 μgBolus mRNA positive control fiber group (see FIG. 9). Additionally, the Schwann cells cultured on the 0.5DSS4h mRNA fibers secreted increased amounts of NT-3 protein 4 and 5 days post-seeding than those cultured on the negative control PLLA fibers (see FIG. 9).

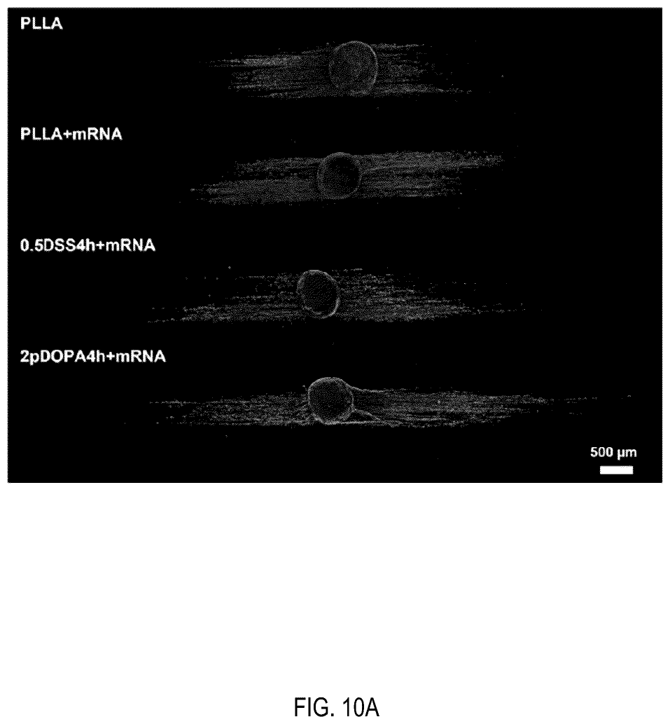

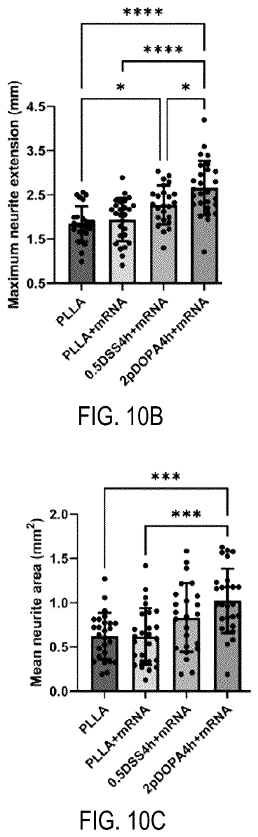

Referring now to FIG. 10A, DRG images were captured via confocal microscopy. The resulting neurite extension and neurite area trends were similar (see FIGS. 10B and 10C). The DRG cultured in the presence of Schwann cells cultured on the 2pDOPA4h mRNA fibers extended longer neurites (2.66±0.62 mm) than those in the presence of Schwann cells on the negative control PLLA fibers (1.84±0.40 mm), PLLA mRNA fibers (1.94±0.49 mm), or 0.5DSS4h mRNA fibers (2.27±0.44 mm) (see FIG. 10B). The DRG cultured in the presence of Schwann cells on the 0.5DSS4h mRNA fibers also extended longer neurites than those in the presence of Schwann cells cultured on the negative control PLLA fibers (see FIG. 10B). The DRG cultured in the presence of Schwann cells on the 2pDOPA4h mRNA fibers extended neurites covered a larger area (1.02±0.37 mm²) than those in the presence of Schwann cells cultured on the negative control PLLA (0.62±0.26 mm²) or PLLA mRNA (0.62±0.32 mm²) (see FIG. 10C).

Previous work reported that whole DRG cultured without additional Schwann cells on aligned, smooth PLLA fibers extended neurites with an average length of 1.77±0.17 mm and area of 0.67±0.10 mm². These values agree well with the neurite extension and area data obtained in these examples from DRG cultured in the presence of Schwann cells cultured on negative control PLLA fibers. This indicates that mRNA delivery and increased Schwann cell secretion of NT-3 likely had a greater impact on DRG neurite outgrowth compared to the presence of additional Schwann cells alone.

The 2pDOPA4h mRNA fiber platform supported the highest Schwann cell secretion of NT-3 protein over 21 days (see FIG. 9). As discussed above, gene delivery from electrospun fibers can be facilitated through the release of the genetic material and uptake by cells local to the material or through direct uptake at the material/cell interface by cells that populate the scaffold. Therefore, the NT-3 mRNA release from the 2pDOPA4h mRNA fibers (see FIG. 7B) could have, in part, improved mRNA delivery to Schwann cells, resulting in increased NT-3 secretion. Additionally, the 2pDOPA4h surface coating may influence electrospun fiber mechanical properties and surface chemistry in a manner that stimulates direct cellular uptake of the lipoplexes immobilized to the fiber surface, improving transfection efficiency and increasing NT-3 secretion. The greatest levels of NT-3 secretion were observed from all experimental groups on day 1 and continued to decrease thereafter. Without wishing to be bound by theory, this may, in part, be due to the increased release of NT-3mRNA at earlier time points and an increased concentration of NT-3 mRNA lipoplexes available on the fiber surface for direct uptake by Schwann cells cultured onto the NT-3mRNA-loaded electrospun fibers. At later time points, however, NT-3mRNA delivery may have relied more on direct cellular uptake of the remaining NT-3mRNA lipoplexes immobilized to the fiber surface.

The DRG neurite outgrowth data reflected those observed in the Schwann cell NT-3 secretion ELISA data. Furthermore, the whole DRG explants cultured in the presence of Schwann cells cultured on the 2pDOPA4h mRNA fiber platforms extended the longest neurites that covered the greatest area (see FIGS. 10A-10B). The 2pDOPA4h mRNA fiber platforms stimulated Schwann cell secretion of NT-3 at a concentration of ˜5000 pg/mL at the 1-day time when DRG explant culture began, and the NT-3 concentration at each subsequent time point fell between 10-106 pg/mL for at least 14 days (see FIG. 9). These findings indicate that the 2pDOPA4h mRNA fibers support Schwann cell secretion of NT-3 at levels capable of stimulating DRG neurite outgrowth.

The 0.5DSS4h- and 2pDOPA4h-coated substrates exhibited increased surface wettability and anionic characteristics compared to the uncoated PLLA fibers while maintaining a high degree of alignment and a fiber diameter of ˜2 μm, which are known to be optimal morphological features for fiber-mediated neural regeneration. Still, 0.5DSS4h- and 2pDOPA4h-coated substrates showed a similar NT-3mRNA loading efficiency and appeared to release NT-3mRNA at an increased rate over the first week compared to the uncoated PLLA fibers. Without wishing to be bound by theory, this observation may, in part, be due to the increased hydrophobicity of the uncoated PLLA fiber group supporting increased nonspecific adsorption of the cationic mRNA lipoplexes compared to the 0.5DSS4h- and 2pDOPA4h-coated fibers. Hydrophobic surfaces facilitate increased nonspecific adsorption from an aqueous solution driven by favorable interfacial energetics, which can be irreversible or less reversible than the specific physical or chemical interactions that we hypothesize occur between the cationic mRNA lipoplexes and the 0.5DSS4h- and 2pDOPA4h-coated fibers.

Methods and systems of the present disclosure are advantageous to provide cells with tunable topographical features and local, sustained, non-viral delivery of mRNA to guide regenerating tissue and enable transient expression of the desired protein. Aligned electrospun fiber networks were functionalized with an anionic layer, e.g., DSS or pDOPA, and further with immobilized cationic NT-3mRNA/JetMESSENGER® lipoplexes to the fiber surface. These functionalized networks upregulate secretion of oligopeptides encoded by their mRNA cargo, e.g., NT-3, and, has been shown to enhance neurite outgrowth from whole DRG explants in vitro. The mRNA complexes of the present disclosure enabled the formation of a monodisperse suspension of cationic mRNA lipoplexes that possessed a similar size and charge observed previously and transfected Schwann cells with high efficiency.

The electrospun fiber scaffolds investigated serve as versatile non-viral mRNA delivery platforms. The mRNA transcript, gene delivery vehicle, and surface coatings can be modified to regulate different desired proteins, optimize transfection efficiency, enable targeted delivery, and tune loading efficiency and release kinetics. Additionally, the electrospun fiber morphological characteristics can be tuned for regeneration of different tissues. Fine-tuning each of these aspects of the electrospun fiber-mediated mRNA delivery platforms enables further tailoring for potential use in various tissue engineering applications.

Exemplary embodiments of electrospun fiber-mediated mRNA delivery platforms for neural regeneration applications were investigated with primary PNS cells in vitro. PLLA mRNA, 0.5DSS4h mRNA, and 2pDOPA4h mRNA aligned electrospun fiber platforms sustained detectable release of NT-3mRNA for at least 28 days. 2pDOPA4h mRNA aligned electrospun fibers increased primary rat Schwann cell secretion of NT-3 for at least 21 days. DRG cultured on uncoated PLLA aligned electrospun fibers in the presence of Schwann cells cultured directly on 2pDOPA4h mRNA aligned electrospun fibers extended the longest neurites and covered the largest area along the PLLA electrospun fibers.

Thus, the 2pDOPA4h mRNA fibers were shown to promote the highest levels of NT-3 protein secretion from Schwann cells over 21 days, which stimulated increased neurite outgrowth from DRG explants compared to the uncoated PLLA control and PLLA mRNA fiber groups, demonstrating the regenerative potential of the NT-3mRNA/JetMESSENGER® lipoplex immobilized pDOPA-coated aligned electrospun fiber platform and use with in vivo peripheral nerve injury treatment.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method of making a functionalized fiber network, comprising:
electrospinning a fiber network, the fiber network including one or more aligned fibers;
preparing mRNA complexes including a cationic transfection portion and an mRNA portion;
immobilizing mRNA complexes on the fiber network to form a functionalized fiber network;
wherein the functionalized fiber network includes an anionic layer disposed between the aligned fibers and the mRNA complexes.

2. The method according to claim 1, wherein the fibers include poly(L-lactic acid), poly(L-lactic acid) derivatives, or combinations thereof.

3. The method according to claim 1, wherein the anionic layer includes dextran sulfate sodium salt (DSS), poly(3,4-dihydroxy-L-phenylalanine) (pDOPA), or combinations thereof.

4. The method according to claim 3, wherein immobilizing mRNA complexes on the fiber network to form a functionalized fiber network includes:
contacting the fiber network with a solution including:
0.5 mg/mL DSS;
2 mg/mL pDOPA,
or combinations thereof.

5. The method according to claim 4, further comprising plasma treating the fiber network prior to immobilizing the mRNA complexes thereon.

6. The method according to claim 1, wherein the cationic transfection portion includes a liposomal transfection agent.

7. The method according to claim 1, wherein the mRNA portion encodes neutrophin-3 (NT-3).

8. The method according to claim 7, wherein the mRNA portion includes Ψ-modified mRNA encoding NT-3.

9. The method according to claim 1, wherein the fiber network is a hollow conduit having a lumen, wherein the mRNA complexes are immobilized within the lumen.

10. The method according to claim 1, wherein the functionalized network further includes one or more additional surface coatings, wherein mRNA complexes are incorporated in the additional surface coating.

11. A functionalized fiber network, comprising:
an electrospun fiber network, the fiber network including one or more aligned fibers;
a plurality of mRNA complexes immobilized on the electrospun fiber network, the mRNA complexes including a cationic transfection portion and an mRNA portion; and
an anionic layer disposed between the aligned fibers and the mRNA complexes.

12. The fiber network according to claim 11, wherein the fiber network includes poly(L-lactic acid), poly(L-lactic acid) derivatives, or combinations thereof.

13. The fiber network according to claim 11, wherein the cationic transfection portion includes a liposomal transfection agent.

14. The fiber network according to claim 11, wherein the mRNA portion encodes neutrophin-3 (NT-3).

15. The fiber network according to claim 14, wherein the mRNA portion includes Ψ-modified mRNA encoding NT-3.

16. The fiber network according to claim 11, wherein the anionic layer includes dextran sulfate sodium salt (DSS), poly(3,4-dihydroxy-L-phenylalanine) (pDOPA), or combinations thereof.

17. The fiber network according to claim 11, wherein the fiber network is a hollow conduit having a lumen, wherein the mRNA complexes are immobilized within the lumen.

18. The fiber network according to claim 11, wherein the functionalized network further includes at least one additional surface coating, wherein mRNA complexes are incorporated in the additional surface coating.

19. A method of delivering a therapeutic agent to a patient, comprising:
electrospinning a fiber network, the fiber network including one or more aligned fibers;
preparing mRNA complexes including a cationic liposomal transfection portion and an mRNA portion including Ψ-modified mRNA encoding NT-3;
immobilizing mRNA complexes on the fiber network to form a functionalized fiber network;
providing the functionalized fiber network as a hollow conduit to a peripheral nerve gap in the patient; and
taking up the mRNA from the functionalized network by one or more cells at or proximate the site of the peripheral nerve gap,
wherein the functionalized fiber network includes an anionic layer disposed between the aligned fibers and the mRNA complexes.

20. The method according to claim 19, wherein the fibers include poly(L-lactic acid), poly(L-lactic acid) derivatives, or combinations thereof and the anionic layer includes dextran sulfate sodium salt (DSS), poly(3,4-dihydroxy-L-phenylalanine) (pDOPA), or combinations thereof.
